(19) United States
(12) Patent Application Publication
(10) Pub. No.: US 2012/0237941 A1
(43) Pub. Date: Sep. 20, 2012

(54) METHOD FOR SCREENING ACTIVE AGENTS THAT STIMULATE THE EXPRESSION OF ARNT2 TO IMPROVE THE SKIN'S BARRIER FUNCTION

(75) Inventor: Elena Fedorova, Whippany, NJ (US)

(73) Assignee: CHANEL PARFUMS BEAUTE, Neuilly sur Seine (FR)

(21) Appl. No.: 13/514,435

(22) PCT Filed: Dec. 3, 2010
(86) PCT No.: PCT/EP2010/068851

§ 371 Date: Jun. 7, 2012

(30) Foreign Application Priority Data
Dec. 7, 2009 (US) 61267154

(51) Int. Cl. C12Q 1/68; G01N 33/566

(52) U.S. Cl. 435/6.12; 435/6.13; 435/7.1

(57) ABSTRACT

A method for screening an active agent intended for preventing or combating the cutaneous signs resulting from a non-pathological impairment of the barrier function, which includes the selection of active agents that stimulate the expression of ARNT2 in cultured human keratinocytes.

The present invention relates to a method for screening an active agent intended for preventing or combating the cutaneous signs resulting from a non-pathological impairment of the barrier function, comprising the selection of active agents that stimulate the expression of ARNT2 in cultured human keratinocytes.

The skin consists mainly of three layers, namely, starting from the uppermost layer, the epidermis, the dermis and the hypodermis.

The epidermis in particular consists of keratinocytes (predominantly), melanocytes (involved in pigmenting the skin) and Langerhans cells. Its function is to protect the body from the external environment and to ensure its integrity, and especially to halt the penetration of microorganisms or chemical substances, to prevent evaporation of the water contained in the skin and to constitute a barrier against external attack and especially against ultraviolet rays (UV).

To do this, keratinocytes undergo a process of proliferation and then of continuous directed maturation during which the keratinocytes located in the basal layer of the epidermis form, at the final stage of their differentiation, corneocytes, which are totally keratinized dead cells in the form of horny sheaths consisting of proteins and lipids such as ceramides. During this differentiation process, intercorneocytic epidermal lipids are also formed and then organized in the form of bilayers (lamellae) in the stratum corneum, and they participate, with the abovementioned horny sheaths, in the barrier function of the epidermis.

The barrier function of the epidermis may, however, be perturbed under certain climatic conditions (for example under the effect of cold and/or the wind) or under the effect of stress or fatigue, especially, thus promoting the penetration of allergens, irritants or microorganisms. These external factors give rise to drying of the skin (the skin loses its permeability, becomes dehydrated and its transepidermal water loss increases), and also to impair the radiance of the complexion and the suppleness of the skin. Impairment of the skin barrier may also promote the appearance of microchapping or microcracks.

Furthermore, a badly formed barrier, resulting from impaired proliferation and differentiation processes, no longer protects the skin against UV radiation or any other type of external attack. The UV rays penetrating the skin may then produce free radicals which may have a detrimental effect on various targets, such as activate collagenases and elastases which are responsible for the degradation of collagen and elastin, respectively, and thus for a decrease in skin elasticity and firmness and the formation of wrinkles.

To prevent or correct the alteration in skin barrier function, it is known practice to apply to the skin cosmetic compositions containing hygroscopic agents, such as sugars or polyols, which are intended to take up the water present in the skin and thus to impede its evaporation. Use has also conventionally been made of fatty substances that allow an occlusive film to be formed on the skin, which contributes towards impeding the evaporation of water. Moreover, these compositions frequently incorporate active agents that act on one or more of the various biological targets involved either in skin turnover processes, in particular in keratinocyte differentiation, epidermal lipid synthesis and corneocyte cohesion, or in the endogenous synthesis of natural moisturizing factor (NMF) constituents of the skin, in particular in the synthesis of proteoglycans.

However, there always remains the need to propose novel cosmetic active agents for reinforcing the skin's barrier function to prevent and/or reduce the sensations of cutaneous discomfort, stinging, tautness, itching, sensations of heating or redness and/or the appearance of microchapping or microcracking and/or the loss of radiance of the complexion or dull complexion and/or to improve the protection of the epidermis against UV.

In addition, given the ever-increasing search by consumers for natural products containing the fewest possible synthetic ingredients, and the increasingly burdensome regulatory constraints on compounds derived from the chemical industry, it would be desirable for these cosmetic active agents to be of plant origin.

Now, the Applicant has, to its credit, shown, unexpectedly, that it is possible to act on a novel biological target, namely the Aryl hydrocarbon Receptor Nuclear Translocator isoform 2 (ARNT2), to combat impairment of the barrier function.

ARNT2 is a member of the basic-helix-loop-helix-Per-Arnt-Sim (bHLH-PAS) family of transcription factors which is involved in adaptation to environmental stress: response to oxidative stress, hypoxia, environmental toxins, and drugs.

The ARNT2 protein is a dimerisation partner for members of the same ARNT group, or a protein belonging to the aryl hydrocarbon receptor (AHR) group. The resulting heterodimers with the sensor proteins then bind regulatory DNA sequences in genes responsive to environmental stimuli. ARNT2 is critical for controlling the activity of gene expression networks in all homeostasis or stress response pathways (E. J. DOUGHERTY et al., Toxicological Sciences, Vol. 103, No. 1, pp. 191-206, 2008; H. SEKINE et al., Journal of Biological Chemistry, Vol. 281, No. 49, pp. 37507-37516, 2006; O. HANKINSON, Toxicological Sciences, Vol. 103, No. 1, pp. 1-3, 2008).

Under hypoxic conditions, Arnt2 forms complexes with hypoxia-inducible factor 1 alpha (HIFα) in the nucleus and this complex binds to hypoxia-responsive elements in enhancers and promoters of oxygen-responsive genes. Increased expression of oxygen-responsive genes resulted in stimulation of Glut1 (glucose transport—increased nutrient and oxygen); VEGF, TGF-beta, PDGF-beta (angiogenesis); TGF-alpha (growth stimulation).

ARNT2 is highly expressed in the nervous system, kidney, and in retina. To the Applicant's knowledge, there are no published data available on ARNT2 expression in skin, let alone in human skin.

It has been shown by S. TAKAGI et al., The Journal of Clinical Investigation, Vol. 112, No. 9 (2003) that ARNT regulates ceramide biosynthesis and is involved in the maintenance of ceramide compositions in the skin, which are crucial for maintaining the epidermal barrier function. Evidence of the expression of ARNT in human and mouse skin, together with its role on epidermal barrier function, has also been brought by S. GENG et al, Journal of Cell Science, Vol. 119, No. 23, pp. 4901-4912 (2006).

However, several authors have stressed that, although ARNT and ARNT2 belong to the same transcriptor factor family and have a very close structure, they are not expressed in the same cells and their biological effects are quite different (see for instance K. HIROSE et al., Molecular and Cellular Biology, Vol. 16, No. 4, pp. 1706-1713, 1996).

Thus, it was not possible, until now, to predict whether ARNT2 may be expressed in human skin cells and which role it may there play, if any.

The Applicant has shown, unexpectedly, that ARNT2 was expressed in human keratinocytes and that it both prevented keratinocytes apoptosis and participated in skin response to UVB. The Applicant has also, to its credit, developed a screening test for selecting active agents, such as plant extracts, acting on this target and which may thus be applied topically on human skin in order to improve skin barrier function.

Moreover, the Applicant has shown that ARNT2 was also expressed in fibroblasts. Thus, compounds that stimulate the expression of ARNT2 could also increase the physiological response to oxidative stress in fibroblasts and thus reduce the oxidative damage caused to cells during lifetime and which are responsible for the impaired structure, appearance and function of aged skin.

One subject of the present invention is thus a method for screening active agents, which are able to prevent or combat the cutaneous signs resulting from non-pathological impairment of the barrier function, comprising the following steps:
a) treating a sample of cultured keratinocytes from a human donor with an active agent, such as a botanical extract;
b) quantifying the expression of ARNT2 in said treated sample, relative to the same cell sample which has not been treated;
c) selecting the active agents that provide for an increase in the expression of ARNT2 relative to the untreated sample.

In this method, the quantification of the expression of ARNT2 may be performed by real time RT-PCR on cultured keratinocytes. In this situation, step c) preferably comprises selecting the active agents that provide for at least a 1.7 fold increase in the gene expression level of ARNT2, compared to the untreated sample where the expression is at 1.

Alternatively, the quantification of the expression of ARNT2 may be performed by immunocytochemical staining on cultured keratinocytes. In such a case, step c) advantageously comprises selecting the active agents that provide for an average intensity the stain obtained by immunocytochemical staining, and assessed visually on a number of random images, which is at least 1.4 time the average intensity assessed for the stain obtained with the untreated sample.

However, any other means for quantifying the expression of ARNT2, for instance by quantifying the production either of messenger RNA of ARNT2 or of ARNT2 protein, may be used without departing from this invention.

The active agents that may be selected according to the invention are advantageously botanical extracts, i.e.

active agents obtained by extraction, using any type of solvent, of any part of a plant such as bark, wood, roots, rhizomes, stalks, leaves, fruit or flowers, for example.

In general, the extraction may be performed on fresh or dried parts of the plant, optionally chopped or ground, in the usual manner. The extraction is generally performed by immersing or gently shaking in one or more polar or apolar solvents or a mixture thereof, at temperatures ranging, for example, from room temperature to 100° C. and advantageously from 30 to 70° C., for a time of about 30 minutes to 12 hours and preferably from 1 to 8 hours. The resulting solution is then preferably filtered so as to remove the insoluble substances of the plant. The solvent is also, where appropriate, removed if it is a volatile solvent, for instance ethanol, methanol or isopropanol.

Alternatively, the botanical extract may be prepared by extraction using a supercritical fluid such as carbon dioxide.

According to still another embodiment, it may be obtained by hydro-distillation, i.e. according to a method including a step for extracting vapour distillation residues, after elimination of the essential oils, by using a polar organic solvent having a polarity index greater than 3.5, possibly mixed with an apolar organic solvent having a polarity index less than 1.

All these extraction methods are common in the field of plant extracts and a person skilled in the art is capable of adjusting the reaction parameters thereof on the basis of his general knowledge.

After this extraction step, a botanical extract is obtained, which may then be subjected to a decolorizing step, especially using active charcoal in the presence of a solvent. The weight of active charcoal is preferably between 0.5% and 50% of the weight of the extract. One or more solvents chosen from water, C₁-C₄ alcohols such as methanol, ethanol or isopropanol, polar organic solvents such as propylene glycol or dipropylene glycol, or any other solvent that is common in the field, may especially be used. The volatile solvents may then be removed under reduced pressure.

The skilled person will be able to prepare various botanical extracts, for example by varying the plants and solvents used. Plant extracts may alternatively be obtained commercially. These active agents can then be subjected to a screening test as described above and in the following examples, so as to determine whether any of these botanical extracts provides for an increase in the gene expression level of ARNT2 and may thus be selected according to this invention.

When it passes the above screening test, the active agent selected according to the invention may be used for cosmetic purposes, to prevent or combat the cutaneous signs resulting from non-pathological impairment of the barrier function, when applied topically to human skin.

The above-mentioned screening method may thus be used for selecting agents capable of protecting skin from signs of drying out, protecting skin from the external environment, especially from the damaging effects of UV rays or from the penetration of toxins, drugs, and chemical substances, and reducing oxidant load in the skin, when applied topically to human skin.

The barrier integrity may especially be measured by corneometry, according to usual techniques that are well known to those skilled in the art.

As a variant, the screening method of this invention may be used for selecting active agents capable of preventing skin photoaging, when applied topically to human skin.

The active agent selected according to the invention, or the composition containing same, are preferably applied to non-pathological dry skin and/or aged skin. They may advantageously be applied to the skin of the face, the neck and possibly the neckline or, as a variant, to any part of the body.

In this regard, the active agent is included in the cosmetic composition, for instance in a proportion of from 0.00001% to 10% by weight, preferably in a proportion of from 0.0001% to 5% by weight and more preferably in a proportion of from 0.001% to 1% by weight relative to the total weight of the composition.

The composition containing this active agent may be applied in the morning and/or in the evening, to the entire face, the neck and optionally the neckline or even the body.

This composition generally comprises, besides the active agent described previously, a physiologically acceptable and preferably cosmetically acceptable medium, i.e. a medium that is suitable for use in contact with human skin without any risk of toxicity, incompatibility, instability or allergic response and especially that does not cause any sensations of discomfort (redness, tautness, stinging, etc.) that are unacceptable to the user.

This medium generally contains water and optionally other solvents such as ethanol.

The composition containing the active agent selected according to the invention may be in any form that is suitable for topical application to the skin and in particular in the form of an oil-in-water, water-in-oil or multiple emulsion (W/O/W or O/W/O), which may optionally be microemulsions or nanoemulsions, or in the form of an aqueous dispersion, a solution, an aqueous gel or an anhydrous composition. It is preferable for this composition to be in the form of an oil-in-water emulsion.

This composition is preferably used as a care and/or cleansing product for facial and/or bodily skin and it may especially be in the form of a fluid, a gel or a mousse, conditioned, for example, in a pump-dispenser bottle, an aerosol or a tube, or in the form of cream conditioned, for example, in a jar. As a variant, it may be in the form of a makeup product and in particular a foundation or a loose or compact powder.

It may contain various adjuvants, such as at least one compound chosen from:
oils, which may be chosen especially from: linear or cyclic, volatile or non-volatile silicone oils, such as polydimethylsiloxanes (dimethicones), polyalkylcyclosiloxanes (cyclomethicones) and polyalkylphenylsiloxanes (phenyl dimethicones); synthetic oils such as fluoro oils, alkylbenzoates and branched hydrocarbons such as polyisobutylene; plant oils and especially soybean oil or jojoba oil; and mineral oils such as liquid petroleum jelly;
waxes such as ozokerite, polyethylene wax, beeswax or carnauba wax;
silicone elastomers obtained especially by reaction, in the presence of a catalyst, of a polysiloxane containing at least one reactive group (especially hydrogen or vinyl) and bearing at least one alkyl group (especially methyl) or phenyl, in a terminal and/or side position, with an organosilicone such as an organohydrogeno-polysiloxane;
surfactants, preferably emulsifying surfactants, whether they are nonionic, anionic, cationic or amphoteric, and in particular fatty acid esters of polyols such as fatty acid esters of glycerol, fatty acid esters of sorbitan, fatty acid esters of polyethylene glycol and fatty acid esters of sucrose; fatty alkyl ethers of polyethylene glycol; alkylpolyglucosides; polysiloxane-modified polyethers; betaine and derivatives thereof; polyquaterniums; ethoxylated fatty alkyl sulfate salts; sulfosuccinates; sarcosinates; alkyl and dialkyl phosphates, and salts thereof; and fatty acid soaps;
co-surfactants such as linear fatty alcohols and in particular cetyl alcohol and stearyl alcohol;
thickeners and/or gelling agents, and in particular crosslinked or non-crosslinked, hydrophilic or amphiphilic homopolymers and copolymers, of acryloylmethylpropanesulfonic acid (AMPS) and/or of acrylamide and/or of acrylic acid and/or of acrylic acid salts or esters; xanthan gum or guar gum; cellulose derivatives; and silicone gums (dimethiconol);
organic screening agents, such as dibenzoylmethane derivatives (including butylmethoxydibenzoylmethane), cinnamic acid derivatives (including ethylhexyl methoxycinnamate), salicylates, para-aminobenzoic acids, β,β′-diphenyl acrylates, benzophenones, benzylidenecamphor derivatives, phenylbenzimidazoles, triazines, phenylbenzotriazoles and anthranilic derivatives;
inorganic screening agents, based on mineral oxides in the form of coated or uncoated pigments or nanopigments, and in particular based on titanium dioxide or zinc oxide;
dyes;
preserving agents;
fillers, and in particular powders with a soft-focus effect, which may be chosen especially from polyamides, silica, talc, mica and fibers (especially polyamide fiber or cellulose fiber);
sequestrants such as EDTA salts;
fragrances;
and mixtures thereof, without this list being limiting.

Examples of such adjuvants are especially mentioned in the CTFA dictionary (International Cosmetic Ingredient Dictionary and Handbook published by The Cosmetic, Toiletry and Fragrance Association, 11th edition, 2006), which describes a wide variety, without limitation, of cosmetic and pharmaceutical ingredients usually used in the skincare industry, that are suitable for use as additional ingredients in the compositions according to the present invention.

The composition containing the active agent selected according to the invention may also provide additional benefits, including calmative or anti-inflammatory activity, bleaching or depigmenting activity and/or cleansing activity.

This composition may also comprise active agents other than those that stimulate the expression of ARNT2, and in particular at least one active agent chosen from: keratolytic agents and in particular β-hydroxy acids such as glycolic acid, lactic acid and citric acid, and esters or salts thereof; β-hydroxy acids such as salicylic acid and derivatives thereof; agents for increasing keratinocyte differentiation and/or cornification, either directly or indirectly by stimulating, for example, the production of β-endorphins, such as extracts of Thermus thermophilus or extracts of bean husks of Theobroma cacao, water-soluble extracts of corn, peptide extracts of Voandzeia substerranea and niacinamide; epidermal lipids and agents for increasing the synthesis of epidermal lipids, either directly or by stimulating certain β-glucosidases that modulate the deglycosylation of lipid precursors such as glucosyl ceramide to ceramides, such as phospholipids, ceramides, lupin protein hydrolyzates and dihydrojasmonic acid derivatives; humectants, such as polyols and in particular glycerol, glycosaminoglycans such as hyaluronic acid, sugars and alkyl esters thereof, amino acids such as glycine, arginine, histidine, alanine, threonine, lysine, glutamic acid, taurine, proline, serine and derivatives thereof, pyrrolidonecarboxylic acid (PCA) and salts thereof, urea and derivatives thereof, ectoin, glucosamine, creatine, choline, betaine, mineral salts such as chlorine, sodium, potassium, calcium, magnesium, zinc, manganese or phosphate salts and humectant synthetic polymers such as methacryloyloxyethylphosphorylcholine homopolymers and copolymers, and glyceryl (meth)acrylate homopolymers and copolymers; antioxidants and/or free-radical scavengers and/or anti-pollution agents, such as tocopherol and esters thereof, ascorbic acid and the alkyl and phosphoryl esters thereof and certain extracts of plants or algae and in particular of Thermus thermophilus; and mixtures thereof, without this list being limiting.

The combination of active agents that stimulate the expression of ARNT2 with one or more of the agents described above makes it possible advantageously to combine in the same formula the effects of these two types of active agent and thus to obtain maximum and long-lasting protection of the skin.

The invention will now be illustrated by the non-limiting examples that follow.

### EXAMPLES

### Example 1

### Test of the Expression Level of the Messenger RNA (mRNA) of the ARNT2 in Normal Human Fibroblasts and Keratinocytes

Protocol:

The expression level of the messenger RNA (mRNA) of the ARNT2 was evaluated on cultured normal human fibroblasts and normal human keratinocytes.

Fibroblasts derived from neonatal foreskins (Cascade Biologics/Invitrogen, Portland, Oreg., USA) are placed in 6-well plates and cultured in DMEM growth culture medium (GIBCO, Invitrogen) supplemented with Fetal bovine serum (GIBCO) and Penicillin-Streptomycin (GIBCO). After culturing for 24 hours in an incubator at 37° C., the almost confluent cells were washed with HBSS buffer (Invitrogen) and used for mRNA extraction.

Keratinocytes derived from neonatal foreskins (Cascade Biologics/Invitrogen, Portland, Oreg., USA) are placed in 6-well plates and cultured in keratinocyte growth culture medium with supplement(Epilife, Invitrogen). After culturing for 24 hours in an incubator at 37° C., the 70% confluent cells were washed with PBS buffer (Invitrogen) and used for mRNA extraction.

The fibroblasts and keratinocytes were cultured for 24 hours. The mRNA was isolated using the Qiagen RNeasy kit and quantified using the QuantIt kit (Invitrogen, CA).

To quantify the expression level of the mRNA of ARNT2 in cell samples, real-time reverse transcription polymerase chain reaction (RT-PCR) was used. The results were normalized relative to the expression of housekeeping genes of these samples. The results were expressed in terms of the number of times of change of expression level of the target gene ARNT2 in two cell types.

The ARNT2 PCR primers were obtained from Applied Biosystems (Applied Biosystems, CA).

Housekeeping gene was GAPDH. The GAPDH PCR primers were obtained from Applied Biosystems (Applied Biosystems, CA).

Reverse transcription was performed using the gene Amp RNA PCR kit (Applied Biosystems) according to the manufacturer's recommendations.

The real-time PCR measurement was performed using the iCYCLER IQ machine (Bio-rad, CA) with Taqman probes.

In all the tests, the cDNA was amplified using a standardized program. Each sample was charged with Taqman master-mix, Taqman primers, probes, and water.

The final amount of cDNA per reaction corresponded to 50 ng of total RNA used for the reverse transcription. The relative quantification of the expression of the target gene was performed using the Pfaffl mathematical model (Pfaffl, M W, Nucleic Acids Res. 29(9), p. E45, 2001).

Results:

The ARNT2 mRNA is expressed both in normal human fibroblasts and normal human keratinocytes. The expression of ARNT2 mRNA in normal fibroblasts is significantly higher than in normal human keratinocytes. Evaluation of expression of the ARNT2 mRNA in fibroblasts and keratinocytes was 53.4 (±3.65) and 1 (±0.2), respectively.

### Example 2

### Test of Stimulation of the Expression of the Messenger RNA (mRNA) of the ARNT2 in Normal Human Keratinocytes with UVB Irradiation

Protocol:

The effect of UVB irradiation on the expression of the messenger RNA (mRNA) of ARNT2 was evaluated on cultured keratinocytes.

Keratinocytes derived from neonatal foreskins (Cascade Biologics/Invitrogen, Portland, Oreg., USA) were placed in 6-well plates and cultured in keratinocyte growth culture medium with supplement (EpiLife, Invitrogen). After culturing for 24 hours in an incubator at 37° C., the 70% confluent cells were washed with PBS buffer (Invitrogen) and then irradiated with UVB using a BioSun instrument (Vilber Lourmat, France) with different doses of UVB and finally incubated for 24 hours in keratinocyte basal culture medium (EpiLife, Invitrogen). The positive results were confirmed using cells from two different donors. The cytotoxicity of the extract was evaluated in human cultured keratinocytes before testing the activity.

A test similar to that of Example 1 was performed.

Results:

Evaluation of expression of the ARNT2 mRNA in keratinocytes from a single donor, irradiated with the doses of UVB 10 mJ/cm²′ 20 mJ/cm², and 30 mJ/cm² was 1.5 (±0.19), 3.6 (±1.25), and 10.9 (±2.37), respectively. In non-treated control keratinocytes expression level was 1.0 (±0.25). The data were confirmed in two donors of keratinocytes.

Increasing the synthesis of ARNT2 by the keratinocytes thus participates in the first means of defense established by the skin to protect itself against UV irradiation. Therefore, compounds which stimulate the expression of ARNT2 in keratinocytes should also protect skin against oxidative stress.

### Example 3

### Effect of ARNT2 Silencing on Keratinocyte Viability in Cell Culture

Keratinocytes derived from neonatal foreskins of a single donor (Invitrogen) were cultured in an incubator at 37° C. with 5% CO₂ in a growth medium suitable for growing keratinocytes (Invitrogen). These keratinocytes were then transfected with a silencer RNA specific for ARNT2 (Ambion, TX) using the transfecting agent NeoFX (Ambion, TX) according to the transfection protocol described by the supplier. Three different siRNAs that suppress ARNT2 gene expression (Ambion, TX) were tested. One siRNA with the best suppression of ARNT2 expression was selected for further experiments. Cells transfected with siRNA to ARNT2, scrambled siRNA (negative control #1) (Ambion, TX), and non-transfected cells (negative control #2) were cultured for 0-5 days. Samples were then analyzed by real time RT-PCR using the same method as that described in Example 1 to confirm knockdown and also using FACS analysis to assess apoptosis.

The effect of ARNT2 silencing on apoptosis was evaluated in keratinocyte cell culture using Annexin V—FITC staining (BD Biosciencies) and FACS analysis. Cells treated with negative control siRNAs and siRNA to ARNT2 were washed with ice-cold PBS and stained in triplicate with Annexin V—FITC following procedures suggested by the manufacturer. After staining cells were analyzed by flow cytometry using FACS Canto II flow cytometer and BD FACSDiva (BD Biosciencies) and FlowJo softwares.

Results:

The results obtained made it possible to demonstrate that inactivation of expression of ARNT2 through the respective silencer RNAs induced a strong reduction in the viability of cultured keratinocytes. Evaluation of the expression of the ARNT2 mRNA in keratinocytes after silencing with siRNA to ARNT2 indicated 60% decrease in ARNT2 expression.

Evaluation of the apoptosis in these keratinocytes from a single donor showed 27.34% (±7.26) increase in apoptosis rate. That in non-treated or treated with scrambled siRNA control keratinocytes was 13.38 (±2.19) and 12.86% (±0.15), respectively. The data were confirmed in two donors of keratinocytes.

It follows from this test that expression of ARNT2 by the keratinocytes is essential for keratinocyte viability.

### Example 4

### Test of Stimulation of the Expression of the Messenger RNA (mRNA) of ARNT2 in Normal Human Keratinocytes with a Synthetic Active Agent

Protocol:

The effect of resveratrol 3,5-diacetate-4′-lipoate, described in Example 1 of WO 2006/134282, on the expression of the mRNA of ARNT2 was evaluated in keratinocytes.

Keratinocytes derived from neonatal foreskins (Invitrogen, CA, USA) were cultured in 6-well plates in keratinocyte culture medium with supplement (EpiLife, Invitrogen). After culturing for 24 hours at 37° C., the 70% confluent cells were washed with PBS buffer (Invitrogen, CA) and incubated with basic keratinocytes culture medium (EpiLife, Invitrogen) containing the active agent to be tested, for 24 hours, at increasing concentrations. The cytotoxicity of the active agent was evaluated in human cultured keratinocytes before testing the activity.

Keratinocytes were treated with various concentrations of active agent in triplicates for 24 hours. The mRNA was isolated using the Qiagen RNeasy kit (Qiagen, CA) and quantified using the QuantIt kit (Invitrogen, CA).

The PCR primers were obtained from Applied Biosystems (Applied Biosystems, CA). The housekeeping gene used was RPLPO.

To quantify the expression of messenger RNA of ARNT2 in a treated sample relative to an untreated sample, real-time polymerase chain amplification (RT-PCR) was performed. The results were normalized relative to the expression of housekeeping gene of these samples. The results were expressed in terms of the fold increase or of decrease in expression of the target gene ARNT2 in the treated sample versus the untreated control.

Reverse transcription was performed using the gene Amp RNA PCR kit (Applied Biosystems) according to the manufacturer's recommendations.

The real-time PCR measurement was performed using the iCYCLER IQ machine (BioRad, CA) with Taqman primers.

In all the tests, the cDNA was amplified using a standardized program. Each sample was incubated with Taqman master-mix, Taqman primers and water. The final amount of cDNA per reaction corresponded to 75 ng of total RNA used for the reverse transcription.

The relative quantification of the expression of the target gene was performed using the Pfaffl mathematical model (Pfaffl, M W, Nucleic Acids Res. 29(9), p. E45, 2001).

The positive results were confirmed using cells from two different donors. The representative data from a single donor are presented in Results.

Results:

The results are given in Table 1 below:

TABLE 1

| | | Stimulation | |
|---|---|---|---|
| | | of ARNT2 | Standard |
| | Concentration⁽¹⁾ | mRNA | deviation |
| Untreated | — | 1.00 | 0.12 |
| keratinocytes | | | |
| Active agent | 0.001% | 1.81 | 0.21 |
| tested | | | |

⁽¹⁾the concentrations of the active agent is expressed as the weight of active agent per weight of preparation

It emerges from this test that the active agent tested makes it possible to significantly stimulate the expression of mRNA of ARNT2 in normal human keratinocytes and can thus be used to maintain skin barrier function.

### Example 5

### Test of Stimulation of the Expression of the Messenger RNA (mRNA) of ARNT2 in Normal Human Keratinocytes with a Botanical Extract

Protocol:

A Vanilla planifolia extract was prepared as described in Example 1 of WO 2007/034042.

The effect of this botanical extract on the expression of the ARNT2 mRNA was evaluated on cultured keratinocytes. A test similar to that of Example 4 was performed.

Keratinocytes were treated with various concentrations of Vanilla planifolia extract in triplicates for 24 hours. The mRNA was isolated using the Qiagen RNeasy kit (Qiagen, CA) and quantified using the QuantIt kit (Invitrogen, CA). The PCR primers were obtained from Applied Biosystems (Applied Biosystems, CA). The housekeeping gene used was GAPDH.

To quantify the expression of messenger RNA of ARNT2 in a treated sample relative to an untreated sample, real-time polymerase chain amplification (RT-PCR) was performed. The results were normalized relative to the expression of housekeeping gene of these samples. The results were expressed in terms of the fold increase or of decrease in expression of the target gene ARNT2 in the treated sample versus the untreated control.

Reverse transcription was performed using the gene Amp RNA PCR kit (Applied Biosystems) according to the manufacturer's recommendations.

The real-time PCR measurement was performed using the iCYCLER IQ machine (BioRad, CA) with Taqman primers.

In all the tests, the cDNA was amplified using a standardized program. Each sample was incubated with Taqman master-mix, Taqman primers and water. The final amount of cDNA per reaction corresponded to 50 ng of total RNA used for the reverse transcription.

The relative quantification of the expression of the target gene was performed using the Pfaffl mathematical model (Pfaffl, M W, Nucleic Acids Res. 29(9), p. E45, 2001).

The positive results were confirmed using cells from two different donors. The representative data from a single donor are presented in Results.

Results:

The results are given in Table 2 below:

TABLE 2

| | | Stimulation | |
|---|---|---|---|
| | | of ARNT2 | Standard |
| | Concentration⁽¹⁾ | mRNA | deviation |
| Untreated | — | 1.00 | 0.00 |
| keratinocytes | | | |
| Vanilla | 0.02% | 2.76 | 0.11 |
| Planifolia | 0.01% | 2.12 | 0.23 |
| | 0.005% | 2.92 | 0.21 |
| | 0.001% | 2.96 | 0.19 |

⁽¹⁾the concentrations of the active agent is expressed as the weight of active agent per weight of preparation

It emerges from this test that the Vanilla Planifolia extracts makes it possible to significantly stimulate the expression of mRNA of ARNT2 in normal human keratinocytes and can thus be used to improve and protect skin barrier function.

### Example 6

### Test of Stimulation of the Expression ARNT2 Protein in Normal Human Keratinocytes with a Botanical Extract

Protocol:

The effect of the botanical extract used in Example 5 on the expression of the ARNT2 protein was evaluated on cultured keratinocytes.

Keratinocytes derived from neonatal foreskins (Invitrogen, CA, USA) were cultivated in 6-well plates containing cover glasses coated with poly-L-ornithine (Sigma, MO) in keratinocyte culture medium with supplement (EpiLife, Invitrogen). After culturing for 24 hours at 37° C., the 70% confluent cells were washed with PBS buffer (Invitrogen, CA) and incubated with basic keratinocytes culture medium (EpiLife, Invitrogen) containing the extract to be tested, for 24 hours. The cytotoxicity of the extract was evaluated in human cultured keratinocytes before testing the activity.

To quantify the expression of the ARNT2 protein in a treated sample relative to an untreated sample, immunocytochemical staining (IC) of cover glasses with cultured keratinocytes from two donors was used. For each donor staining was performed in triplicates, with primary rabbit anti-human ARNT2 antibodies (Santa Cruz, Calif.) and secondary goat anti-rabbit antibodies (Lab Vision Corporation, CA). The staining was visualized with AEC system (Lab Vision Corporation, CA). The extent of staining was assessed on thirty random images for each experimental condition, and a visual assessment of the staining was made using a scale from 1 to 5, with 1 being the least intense and 5 being the most intense. The significance of the difference between mean values was assessed using unpaired t test.

Results:

Evaluation of ARNT2 staining in keratinocytes treated with 0.05% Vanilla Planifolia was 4.52 (±0.51), in keratinocytes treated with 0.025% Vanilla Planifolia it was 3.93 (±0.83) and in non-treated control keratinocytes it was 1.52 (±0.70). The difference of ARNT2 staining in untreated and treated keratinocytes was significant (p<0.0002). This demonstrates that the amount of ARNT2 protein increases with 0.05% and 0.025% Vanilla Planifolia treatments.

The results are given in Table 3 below:

TABLE 3

| | | Stimulation | |
|---|---|---|---|
| | | of ARNT2 | Standard |
| | Concentration⁽¹⁾ | protein | deviation |
| Untreated | — | 1.52 | 0.70 |
| keratinocytes | | | |
| Vanilla | 0.05% | 4.52 | 0.51 |
| Planifolia | 0.025% | 3.93 | 0.71 |

⁽¹⁾the concentrations of the extract is expressed as the weight of crude extract per weight of preparation

It emerges from this test that Vanilla Planifolia extracts make it possible to stimulate the expression of ARNT2 protein in normal human keratinocytes and may thus be used to protect and improve skin barrier function.

### Example 7

### Test of Stimulation of the Expression ARNT2 Protein in Normal Human Keratinocytes with a Synthetic Active Agent

Protocol:

The effect of the active agent used in Example 4 on the expression of the ARNT2 protein was evaluated in keratinocytes. A test similar to that of Example 6 was performed.

The positive results were confirmed using cells from two different donors. The representative data from single donor are presented in Results.

Results:

Evaluation of ARNT2 staining in treated keratinocytes was 2.22 (±0.64) and that in non-treated control keratinocytes was 1.52 (±0.70). The difference of ARNT2 staining in untreated and treated keratinocytes was significant (p=0.0002).

The results are given in Table 4 below:

TABLE 4

| | | Stimulation | |
|---|---|---|---|
| | | of ARNT2 | Standard |
| | Concentration⁽¹⁾ | protein | deviation |
| Untreated | — | 1.56 | 0.73 |
| keratinocytes | | | |
| Active agent | 0.001% | 2.22 | 0.64 |
| tested | | | |

⁽¹⁾the concentrations of the active agent is expressed as the weight of active agent per weight of preparation

It emerges from this test that the active agent tested significantly stimulates the expression of ARNT2 in normal human keratinocytes and may thus be used to maintain skin barrier function and to protect the skin against the damaging effects of UV irradiation.

### Example 8

### Cosmetic Composition

The following composition may be prepared in a manner that is conventional for those skilled in the art. The amounts indicated below are expressed as weight percentages. The ingredients in upper case are identified in accordance with the INCI name.

| | |
|---|---|
| Tetrasodium EDTA | 0.05% |
| POLYGLYCERYL METHACRYLATE & | 5.00% |
| PROPYLENE GLYCOL⁽¹⁾ | |
| Glycerol | 6.00% |
| Aqueous-phase gelling agents | 5.50% |
| Nonionic emulsifiers | 4.00% |
| Cetearyl alcohol | 2.00% |
| Emollients | 17.00% |
| Tocopheryl acetate | 0.50% |
| Preserving agents | 2.20% |
| Botanical extract⁽²⁾ | 0.05% |
| Sodium hyaluronate | 5.00% |
| Fragrance | qs |
| Dyes | qs |
| Water | qs 100.00% |

⁽¹⁾LUBRAJEL MS ® from Guardian Laboratories
⁽²⁾obtained by screening various plant extracts on the test disclosed in Example 4 or 5

This composition, in the form of an oil-in-water emulsion, may be applied daily, morning and/or evening, to facial skin to moisturize it and make it supple, smooth and luminous.

1. A method for screening active agents, which are able to prevent or combat the cutaneous signs resulting from non-pathological impairment of the barrier function, comprising the following steps:
a) treating a sample of cultured keratinocytes from a human donor with an active agent, such as a botanical extract;
b) quantifying the expression of ARNT2 in said treated sample, relative to the same cell sample which has not been treated;
c) selecting the active agents that provide for an increase in the expression of ARNT2 relative to the untreated sample.

2. The method according to claim 1, characterized in that the quantification of the expression of ARNT2 is performed by RT-PCR.

3. The method according to claim 2, characterized in that step c) comprises selecting the active agents that provide for at least a 1.7 fold increase in the gene expression level of ARNT2, compared to the untreated sample where the expression is at 1.

4. The method according to claim 1, characterized in that the quantification of the expression of ARNT2 is performed by immunocytochemical staining.

5. The method according to claim 4, characterized in that step c) comprises selecting the active agents that provide for an average intensity the stain obtained by immunocytochemical staining, and assessed visually on a number of random images, which is at least 1.4 time the average intensity assessed for the stain obtained with the untreated sample.

6. The method according to claim 1, wherein the selected active agents are capable of preventing or combating the cutaneous signs resulting from non-pathological impairment of the barrier function, when applied topically to human skin.

7. The method according to claim 1, wherein the selected active agents are capable of protecting skin from signs of drying out, protecting skin from the external environment, especially from the damaging effects of UV rays or from the penetration of toxins, drugs, and chemical substances, and reducing oxidant load in the skin, when applied topically to human skin.

8. The method according to claim 7, characterized in that said signs are chosen from: skin roughness, the loss of radiance of the complexion and/or the loss of suppleness of the skin.

9. The method according to claim 1, wherein the selected active agents are capable of preventing skin photoaging, when applied topically to human skin.
